(19) United States
(12) Patent Application Publication
(10) Pub. No.: US 2008/0133017 A1
(43) Pub. Date: Jun. 5, 2008

(54) Assembled Prosthesis Such as a Disc

(75) Inventors: Mordechay Beyar, Caesarea (IL); Oren Globerman, Kfar-Shemaryahu (IL)

Correspondence Address: Martin D. Moynihan;PRTSI, P.O.Box 16446, Arlington, VA, 22215, US

(73) Assignee: Disc-o- Tech Medical Technology, Herzlia pituach (IL)

(21) Appl. No.: 11/667,759

(22) PCT Filed: Nov. 15, 2005
(86) PCT No.: PCT/IL05/01198

§ 371 Date: Oct. 29, 2007

Related U.S. Application Data
(60) Provisional application No. 60/627,141, filed on Nov. 15, 2004.

(51) Int. Cl. A61F 2/44

(52) U.S. Cl. 623/17.16; 623/17.14; 623/17.11

(57) ABSTRACT

An artificial disc device, comprising a plurality of interconnected elements, adapted to be assembled in situ.

### RELATED APPLICATIONS

The present application is a US National Phase of PCT Application No. PCT/IL2005/001198, filed on Nov. 15, 2005, published as WO 2006/051547. This application is based upon and claims the benefit under 35 USC 119(e) of U.S. provisional patent application Ser. No. 60/627,141, filed Nov. 15, 2004, the disclosures of which are incorporated herein by reference in their entirety.

### FIELD OF INVENTION

The present invention relates to a prosthesis device, for example one used in a minimally invasive method for replacing a damaged intervertebral disc.

### BACKGROUND OF THE INVENTION

The vertebral bodies of the spine are connected to one another by a disc. The intervertebral discs give the column its flexibility and mobility. Each intervertebral disc comprises a nucleus pulposus (“nucleus”) surrounded by the annulus fibrosus (“annulus”). The latter binds together adjacent vertebrae, and is composed of multiple and overlapping layers of concentric, collagen fiber rings. Each layer comprises fibers arranged in a right and left diagonal orientation. This unique arrangement contributes to a bi-directional torsional motion resistance. The nucleus is located inside the annulus and has about 85% water content, which decreases with age. The nucleus moves (and bulges) within the annulus when force is exerted on adjacent vertebrae (during, for example, vertebral column flexion, extension or bending sideways).

Trauma, spinal diseases (such as degenerative disc disease) and normal aging processes may damage the intervertebral disc. For example, a weakening or tear of the annulus fibers may lead to disc herniation, as the nucleus extrudes out of its location inside the annulus ring. This mass extrusion can mechanically press on neighboring spinal nerves, resulting in back pain, radiation pain, loss of muscle control and even paralysis.

Various treatments for a damaged disc are available. Where conservative treatment such as pain reduction by medication fails, surgical treatment usually becomes necessary. Such surgery may include removing the herniated disc (discectomy) and decompressing the involved nerve. Although discectomy is effective in relieving significant radicular pain, in general pain recurs in direct proportion to time from surgery. The only temporary success of discetomy is probably due to continuation of the degenerative process, segmental instability and spinal stenosis. A decrease in the disc height is quite common after discectomy, and might result in size decrease of the neural foramina, alteration of facet loading and function, as well as adverse effect on sagittal balance. In addition, disc height reduction increases intraarticular pressure and predisposes individuals to develop hypertrophic changes of the articular processes (Vincent C., Traynelis M.D., Spinal Arthroplasty, Neurosurg Focus 13 (2), 2002, 2002 American Association of Neurological Surgeons, Medscape).

Alternative surgery comprises immobilization of the involved segment, during which the disc is removed and adjacent vertebrae are connected/fused together, for example by bone grafting, an interbody fusion device and/or pedicle screws. In general, the procedure relieves the motion-induced discogenic pain, preserves sagittal balance, halts further degeneration of the involved level, and can restore disc space height. However, long-term results indicate that numerous patients develop recurrent symptoms years after surgery. In addition, the procedure causes permanent loss of function and mobility of the involved segment. It also perturbs the biomechanics of adjacent vertebral levels, and may induce more rapid degeneration of adjacent segments. Furthermore, most of the devices intended for implantation during vertebral fusion require highly invasive surgical technique, resulting in damaging of the surrounding tissue, including bone structure sacrifice that may compromise spinal stability.

Another option for treating a damaged disc is implantation of a total disc prosthesis or of a nucleus prosthesis. Disc nucleus replacement is performed where nucleus has undergone significant degeneration while annulus is relatively healthy. The artificial disc offers several potential advantages over the spinal fusion method, including enhanced clinical success rate (pain reduction), and avoiding a premature degeneration at adjacent level. There are currently artificial total disc systems that are cleared for marketing or in different phases of development and clinical trials—such as the Charite Artificial Disc, by DePuy Spine Group Inc.; the Prodisc, by Spine Solutions/Synthes; the Maveric, by Medtronic Sofamor Danek; and the FlexiCore, by SpineCore—all of which are implanted in a relative invasive surgical techniques, normally in an open anterior approach, which require a large insertion aperture and may lead to risks and complications thereof.

### SUMMARY OF THE INVENTION

An aspect of some embodiments of the invention relates to assembling a prosthesis from parts, in situ. In an exemplary embodiment of the invention, the prosthesis is an intervertebral disc. In an exemplary embodiment of the invention, the disc is formed of interlocking parts. In an exemplary embodiment of the invention, the parts have an orientation with a maximal cross-section of less than 12×12 or 10×10 mm. Optionally, the parts include at least one channel for receiving a matching protrusion in another part. Optionally, the parts include at least one anchor point used to anchor the part relative to a delivery system and/or a partially assembled prosthesis, during assembly. Optionally, following assembly, at least some of the parts are laterally situated with respect to each other when installed in the patient.

In an exemplary embodiment of the invention, the parts interlock one to the side of the other.

In an exemplary embodiment of the invention, the parts interlock as they are assembled in situ. Optionally, the interlocking is rigid interlocking which prevents relative motion in any direction. Optionally, a small freedom of motion remains after interlocking, for example representing less than 3 mm, 2 mm, 1 mm, or 0.5 mm of motion. This may be provided by providing spaces between the interlocking elements. Optionally, 1, 2, 3, 4 or more degrees of motion remain to the interconnection between the parts. Optionally, the freedom is limited as noted herein. Alternatively it may not be limited by the interlock mechanism itself. Alternatively, there is no substantial freedom of motion in any direction once interlocked.

In an exemplary embodiment of the invention, each part (in some embodiments formed of two or more disjoint sections) is mounted on a delivery element and the delivery elements guide the parts into assembly.

Optionally, the prosthesis includes one or more tissue engaging elements. In an exemplary embodiment of the invention, a delivery element is used to at least guide the deployment of one or more tissue engaging elements that lock the prosthesis to nearby tissue.

A potential advantage of in situ assembly is the ability to implant the artificial disc using a minimally invasive procedure, through a relatively minor port into the body, and thus potentially reducing the damage to surrounding tissue and spinal stability, simplifying the operation and/or facilitating recovery.

In an exemplary embodiment of the invention, the disc is formed of two horizontal situated plates and an optional core/spacer element between them. In an exemplary embodiment of the invention, the plates are designed to be parallel to each other, in situ. Alternatively, at least one of the plates is oblique (e.g., with a trapezoid cross-section) or designed to be oriented obliquely relative to the other plate, for example, to comply with lumbar lordosis.

In an exemplary embodiment of the invention, the plates and/or the core/spacer element are made of biocompatible metal, such as, for example, implant grade stainless steel, titanium, cobalt chromium and/or combinations thereof. Alternatively or additionally, at least one of prosthesis components is constructed from biocompatible ceramics, such as, for example, alumina ceramics. Alternatively or additionally, at least one of the prosthesis components is constructed from biocompatible polymer, such as, for instance, polyurethane, polycarbonate urethane, polyethylene and/or silicone.

In an exemplary embodiment of the invention, the core/spacer element is an integral part of one of the plates, for example the lower plate, and the upper plate, incorporates a concavity/recess matching the core/spacer element. Alternatively, the core/spacer element is a separate component which is optionally assembled to be mounted on least one of the plates, in situ. In an exemplary embodiment of the invention, the core/spacer element is inserted after the prosthesis plates are inserted and positioned. Optionally, the core/spacer element is pliable enough to enable its entire insertion in one piece, for example by its shrinking or contracting during introduction; following insertion and positioning between the prosthesis plates, the core/spacer element regains its original diameter and configuration.

In an exemplary embodiment of the invention, the plates are divided into slices that are substantially perpendicular to the plate surfaces, so that each plate comprises, for example, 3, 4, 5, 6, 7 or 8 slices, depending on disc size and location of implantation. Optionally, the plates are sliced in the sagittal plane (anterior-posterior) so that when inside the body, the boundaries between the slices/parts are substantially perpendicular to the vertebral plates. Optionally, the average angle is between 30 degrees and 90 degrees. It should be noted that the boundary is, in some embodiments, not straight, for example due to interlocking element design. Optionally, parallel slices of the two plates are similar or identical in size and configuration. Each plate slice may have a maximum diameter of, for example, 3 mm, 4 mm, 5 mm, 6 mm, 7 mm, 8 mm, 10 mm, 12 mm or more or intermediate or smaller diameter, and different slices of the same plate may have different diameters. In an exemplary embodiment of the invention, the diameter is defined as the maximum cross-sectional diameter in an orientation where this diameter is minimal (e.g., an orientation along which it is implanted).

In an exemplary embodiment of the invention, each prosthesis portion comprises corresponding slices of the two plates, which are inserted together, loaded on a single delivery system/inserter. In an exemplary embodiment of the invention, the assembly of adjacent portions is enabled by a locking mechanism, for example of a male-female type, which may also enable insertion of one prosthesis portion over the side of its adjacent, previously inserted, portion. In additional embodiment of the invention, the locking mechanism between adjacent portions of the prosthesis prevents relative motion between prosthesis portions in all planes.

In an exemplary embodiment of the invention, the prosthesis comprises six portions (e.g., portions of the device which are formed of parts of the disc that are in the form of slices), and each is optionally introduced into the intervertebral space via a port aligned with an expected position of the last portion to be inserted (e.g., the fifth portion as explained below. In an exemplary embodiment of the invention, the prosthesis portions are inserted in a consecutive order, from the first, lateral portion, with the exception that the sixth portion (positioned in the other end) is introduced prior to the fifth portion. Alternatively, there is a different number of prosthesis portions, and/or sequence of portions insertion, and/or location of insertion aperture.

In an exemplary embodiment of the invention, the proximal end of the delivery system of each portion of plates comprises an external alignment section, which is located out of the patient body while the implanted portion is introduced into the intervertebral space. In an exemplary embodiment of the invention, the alignment sections are designed to align in a manner matching the internal interlocking, thus possibly allowing the process of assembly inside the body to be indicated or mirrored outside the body, for clear, non-imaging, reference. Optionally, the trial sections are replica of the prosthesis portions, thus having identical design and dimensions.

In an exemplary embodiment of the invention, the design of the artificial disc enables a relative motion between the two plates, to restore motion and stability of the affected spinal segment. In additional embodiment of the invention, the design of the artificial disc allows for disc height restoration, for example by providing a vertical dimension similar to or greater than that of a natural disc being replaced.

In an exemplary embodiment of the invention, the plates are attached to at least one of the adjacent vertebrae. In an exemplary embodiment of the invention, the attachment is enabled by at least one protrusion/spike which protrudes from the external surface of the plate. In an exemplary embodiment of the invention, during prosthesis insertion the spikes are hidden and the external surface of the plates is relative smooth/flat. Following insertion of all the prosthesis portions and their assembly, the protrusions are projected to penetrate the adjacent vertebrae endplates by a designated mechanism, for example, to prevent possible prosthesis migration. In an alternative embodiment, protrusions penetrate the adjacent vertebrae endplates following insertion of each portion of the plates, before the entire prosthesis is assembled. Optionally, the “activation” of the protrusions is caused and/or performed by a delivery system of the prosthesis. In an exemplary embodiment of the invention, portions of prosthesis are inserted into the disc space without said protrusions, and only after all said portions are inserted, or after each portion is inserted, the protrusions are introduced into the prosthesis portions and vertebral endplates.

In an exemplary embodiment of the invention, at least one of the two metal endplates are coated, for instance with hydroxyapatite (HA) or titanium plasma spray, to enhance osteointegration and/or improve plates connection to adjacent vertebrae.

In an exemplary embodiment of the invention, the insertion process comprises removal of disc material and insertion of a total disc prosthesis, optionally at one or two sides of a dura (e.g., some of disc portions inserted from one side and some from the other). Optionally, the insertion is from the back of the patient. In one embodiment of the invention, the prosthesis is introduced in several small portions, assembled in situ, and then is optionally connected to at least one of the adjacent vertebral endplates.

In another embodiment of the invention, the implant is inserted inside the disc space percutaneously, in a posterior or postero-lateral approach. Alternatively, the implant is inserted using a lateral approach. Alternatively, introduction of said implant is performed using a minimally invasive open posterior or anterior approach.

In an exemplary embodiment of the invention, the procedure is monitored by CT scanning, fluoroscopy and/or other imaging methods. In one embodiment of the invention, the implant is constructed from radiopaque material, to enable its tracking during and after surgery. Alternatively or additionally, a radiopaque probe/marker is added to the implant, for example by insertion of a marker unit into an aperture formed in the prosthesis. In an exemplary embodiment of the invention, the radio-opaque markers are arranged so that correct alignment and/or locking of the prosthesis portions can be seen when imaging.

In an exemplary embodiment of the invention, an instrumentation set is provided, intended to assist minimal invasive deployment and/or assembly of a prosthesis as described herein.

In an exemplary embodiment of the invention, for example if removal or replacement of the disc prosthesis is indicated, the prosthesis is removed using a minimally invasive procedure. In an exemplary embodiment of the invention, each portion of prosthesis plates is extracted separately, using a designated extractor, through a relative small port. Optionally, the delivery system is used for prosthesis extraction as well, for example, each delivery element is attached to a part of the prosthesis and then is used to pull it out. Optionally, the attachment is under fluoroscopy.

An aspect of some embodiments of the invention relates to in situ assembly of an implant, in which elements are added to an existing assembly one at a time. In an exemplary embodiment of the invention, the elements are aligned using a delivery system associated with each element. Optionally, as an element becomes part of the assembly, its delivery system is removed. Optionally, at any given time at most two delivery systems are in place, that of an assembled element and that of an element being assembled.

An aspect of some embodiments of the invention relates to a set of delivery elements designed to align such that elements delivered using the delivery elements will interlock in a corresponding manner. Optionally, the delivery systems are designed to be removed as the delivered elements interlock.

An aspect of some embodiments of the invention relates to a disc prosthesis including at least one deploying protrusion, which protrusion deploys only after the disc is inside the body. Optionally, the protrusion is kept from early deployment by a restraint which forms part of the prosthesis.

An aspect of some embodiments of the invention relates to implanting a disc prosthesis from a posterior aspect, for example from the back of a patient while bypassing the spinal column. Optionally, the implantation is via a narrow port into the body. Optionally, the port is smaller than a deployed prosthesis. In an exemplary embodiment of the invention, the prosthesis is assembled in situ. In some embodiments, part of the prosthesis is inserted from a port on one side of the dura of the spine and some from a port on another side.

There is thus provided in accordance with an exemplary embodiment of the invention, an artificial disc device, comprising a plurality of interconnected elements, adapted to be assembled in situ. Optionally, the elements are adapted to interconnect one to the side of the other. Alternatively or additionally, said elements are adapted to interlock at least in pairs. Optionally, the device comprises two opposing plates, each adapted to be placed near an opposite vertebral plate, along the axis of the spinal column of a patient into whom it is inserted. Optionally, said plates define a ball joint between them. Optionally, said ball joint comprises a ball and socket and wherein said ball is integral to one of said plates. Alternatively, said ball is not integral with either plate.

In an exemplary embodiment of the invention, said device comprises at least 4 separate elements, assembled into said device.

In an exemplary embodiment of the invention, the device comprises at least one protrusion adapted to selectively deploy and engage a vertebral plate. Optionally, said protrusion is adapted to deploy when said deployment is complete. Alternatively, said protrusion self-deploys.

In an exemplary embodiment of the invention, at least a plurality of said elements are used to assemble each of said plates.

In an exemplary embodiment of the invention, said interlocking elements, after said interlocking have some freedom of relative motion. Optionally, said elements interlock using a self-locking mechanism.

In an exemplary embodiment of the invention, said interlocking elements comprise alignment portions in the form of matching parts. Optionally, said alignment portions comprise male-female matching portions.

In an exemplary embodiment of the invention, the device comprises a delivery system attached to at least two of said elements. Optionally, said delivery system comprises at least two delivery elements, each attached to a different interlocking element. Optionally, said delivery elements are configured to align with each other in a manner that matches an alignment of said elements to which they are attached. Alternatively or additionally, a single delivery element is attached to two corresponding elements form two non-interlocking parts of said device.

In an exemplary embodiment of the invention, said plates are formed of metal.

In an exemplary embodiment of the invention, the device has an osteointegrating outer layer.

There is also provided in accordance with an exemplary embodiment of the invention, a method of deploying a disc prosthesis, comprising:

forming a path to a disc area;

providing a first disc portion to said disc area;

providing a second disc portion to said disc area; and

assembling said second disc portion to said first disc portion at said disc area.

Optionally, a last provided disc portion interlocks with two previously provided adjacent disc portions. Alternatively or additionally, said providing a first disc portion and said providing a second disc portion comprises aligning delivery elements each associated with one of said disc portions. Optionally, the method comprises removing a delivery element associated with said first portion, but not an element associated with said second portion, when a third disc portion is provided using a third delivery element.

In an exemplary embodiment of the invention, the method comprises releasing at least one protrusion to contact an adjacent vertebral plate. Optionally, said release is after performed said assembly.

In an exemplary embodiment of the invention, said release is performed during said assembly.

In an exemplary embodiment of the invention, the method comprises assembling at least one vertebral plate engager to at least one of said disc portions, after provision of said at least one disc portion into a body.

In an exemplary embodiment of the invention, said path is to a side of a dura.

In an exemplary embodiment of the invention, said path is for posterior access.

In an exemplary embodiment of the invention, said path has a maximum diameter of less than 20 mm.

In an exemplary embodiment of the invention, said path has a maximum diameter of less than 15 mm.

In an exemplary embodiment of the invention, forming said path comprises inserting a cannula for said providing.

In an exemplary embodiment of the invention, assembling comprises interlocking.

In an exemplary embodiment of the invention, said portions each comprises a plurality of non-interlocking disc parts.

There is also provided in accordance with an exemplary embodiment of the invention, a disc prosthesis, comprising:

at least one plate adapted to contact a vertebral plate; and

at least one protrusion adapted to extend towards said plate after positioning of said plate. Optionally, said protrusion self deploys after said disc is assembled in situ. Alternatively, said at least one protrusion is allowed to deploy by release thereof.

In an exemplary embodiment of the invention, the prosthesis comprises a deployment limiting element integral to said prosthesis.

There is also provided in accordance with an exemplary embodiment of the invention, a delivery system for assembling a device in situ, comprising:

a first elongate element adapted to attach to a first device portion and including a first external indicator section adapted to remain outside a body; and

a second elongate element adapted to attach to a second device portion and including a second external indicator section adapted to remain outside a body,

wherein said external indicator sections are configured to interrelate in a manner indicating an interrelation of said device portions. Optionally, said device is a disc prosthesis.

In an exemplary embodiment of the invention, at least one of said portions comprises disjoint sub-portions of said device.

In an exemplary embodiment of the invention, said system comprises at least three elements and wherein said indicator sections are configured to allow interconnection in a single manner only.

### BRIEF DESCRIPTION OF THE FIGURES

Some exemplary embodiments of the invention will be further described with reference to the accompanied drawings, in which same number designations are maintained throughout the figures for each element and in which:

FIG. 1 is a general illustration of a disc prosthesis, in accordance with some exemplary embodiments of the present invention;

FIG. 2 illustrates the inferior (lower) plate of the disc prosthesis of FIG. 1, in accordance with some exemplary embodiments of the present invention;

FIG. 3 illustrates the superior (upper) plate of the disc prosthesis of FIG. 1, in accordance with some exemplary embodiments of the present invention;

FIGS. 4A-4D describe an option for prosthesis portions insertion sequence and manner, in accordance with some exemplary embodiments of the present invention;

FIG. 5A illustrates the last prosthesis portion to be introduced, in accordance with some exemplary embodiments of the present invention;

FIGS. 5B-5D illustrate, in a simplified manner, a locking mechanism for interlocking portion of the prosthesis of FIG. 1, in accordance with an exemplary embodiment of the invention;

FIGS. 6A-6C illustrate a manner of connection between the prosthesis and adjacent vertebrae, in accordance with some exemplary embodiments of the present invention;

FIG. 7 illustrates a different manner of connection between the prosthesis and adjacent vertebrae, in accordance with some exemplary embodiments of the present invention; and

FIG. 8 is a flowchart of an exemplary method of prosthesis insertion, in accordance with an exemplary embodiment of the invention.

### DETAILED DESCRIPTION OF EXEMPLARY EMBODIMENTS

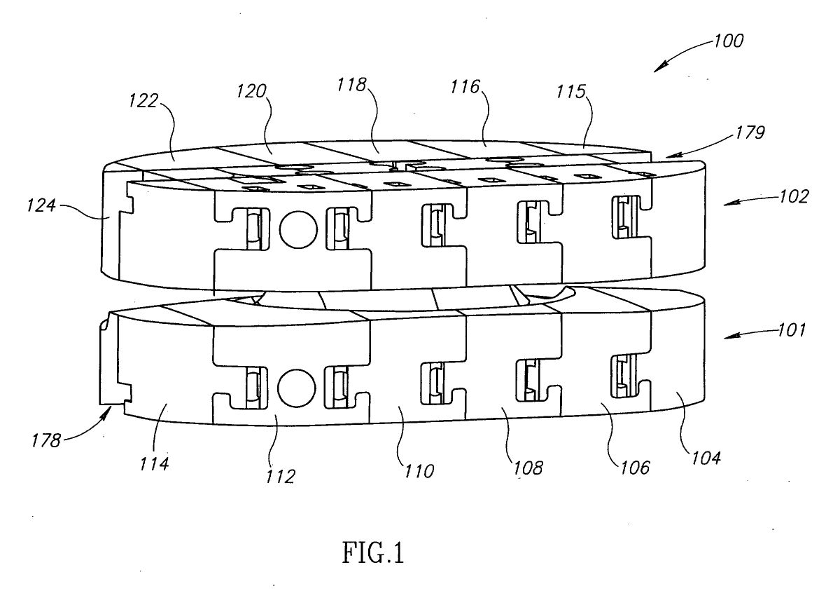

FIG. 1 illustrates a disc prosthesis 100, comprising an inferior plate 101 and a superior plate 102, in accordance with an exemplary embodiment of the invention. The two plates 101, 102 have a same circumference/contour line, although different dimensions and/or shapes for each of the two plates 101, 102 may be provided in some embodiments of the invention. Each plate 101, 102 is composed of six slices 104-114, 115-124, respectively. Other number of slices may be provided and, in general, the number of plate slices and their cross-sections may depend on disc size and/or the disc's location. The plates 101, 102 may be connected to their adjacent vertebral endplates by protrusions (not shown in FIG. 1), for example protrusions that project from a slot (or a windowing element) 178, 179 in each plate 101, 102, respectively, toward endplates of adjacent vertebrae and penetrate them following prosthesis insertion, for example, to prevent prosthesis migration.

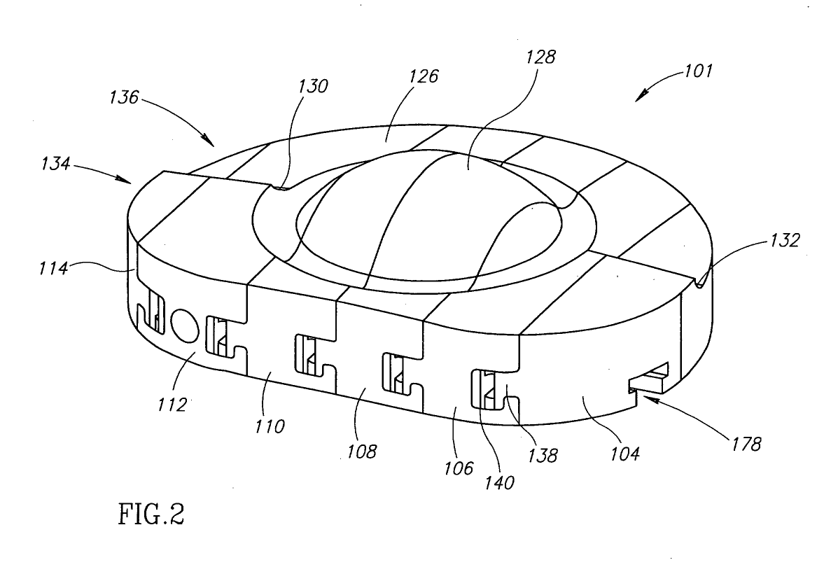

FIG. 2 shows lower plate 101 of disc prosthesis 100, in accordance with an exemplary embodiment of the invention. Approximately at its center, the upper surface 126 of the plate 101 is optionally convex, forming a core element 128, which serves as a ball of a ball-and-socket-like joint. Optionally, core element 128 is located closer to the plate posterior edge than to the anterior one, to provide for a more posterior center of rotation. Other adaptations to anatomy and/or functions of discs may be provided. In an alternative embodiment, the ball is provided separately, for example as a hard rubber ball compressed to fit through a lumen of a cannula and released into a joint area defined by plates 101 and 102. Optionally, the ball is replaced if it wears down.

Plate 101 upper surface 126 optionally includes two lateral steps 130, 132 that create a plate 101 with a higher posterior section 134 relative to an anterior section 136 thereof. This structure is optionally used to allow for different motion angles in various directions (e.g., larger range of motion for forward bending (flexion) than for extension). As shown, plate 101 comprises six slices 104-114. Optionally, each slice 104-114 comprises at least one male 138 and/or female 140 connecting feature at one or two sides thereof, which interlock adjacent slices 104-114 to each other. In an exemplary embodiment of the invention, the connecting features are sliding features. As noted above and shown below, the plate external (lower) surface optionally comprises one or more slots 178 (or other opening), through which prosthesis spikes protrude and penetrate adjacent endplate (not shown in FIG. 2).

Optionally, the slice design of the two plates (or the ball and the matching depression) is selected to not match, so as to reduce wear caused by matching cracks between the slices aligning. Optionally, the orientation of the cracks is not the same. Optionally, instead of a sliding relationship between slices, as described below, a different interlocking is used, so that the cracks are not straight, but are, for example, curved. This can be done, for example, if the slices define a curved rail on one slice and a following peg on the other slice. Straight slice edges may be stronger/more rigid.

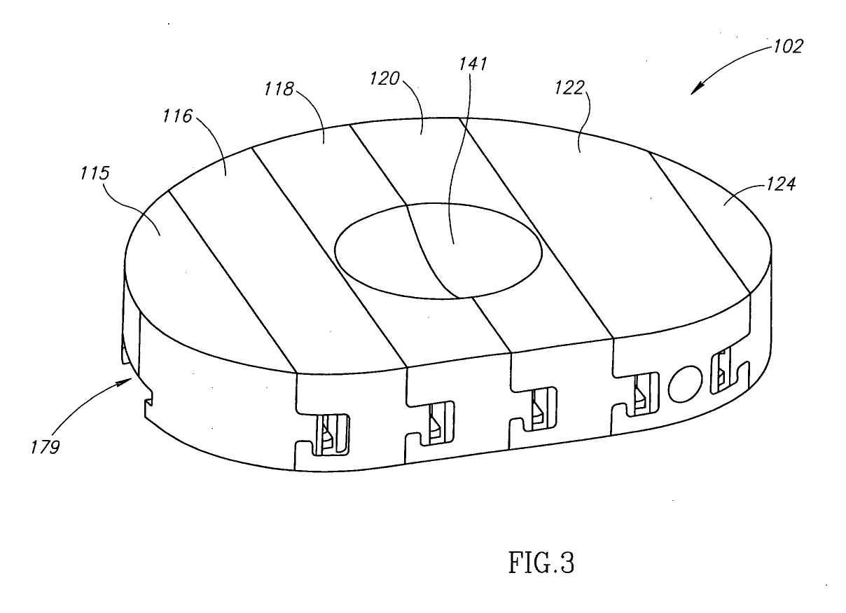

FIG. 3 illustrates a superior (upper) plate 102 of disc prosthesis 100, in accordance with an exemplary embodiment of the invention. For clarity, superior plate 102 is presented so that its lower surface is facing up. The plate 102 optionally comprises approximately at its center a recess/socket 141 that matches the inferior plate convex 128, together forming a joint. Upper plate 102 optionally comprises six slices 115-124. Optionally, the slice sections of the two plates (101, 102) correspond to each other. Optionally, each slice 115-124 has at least one male and/or female connecting design to lock adjacent slices. Plate external (upper) surface optionally comprises at least one slot or other opening 179, through which prosthesis spikes optionally protrude and penetrate adjacent endplate (not shown in FIG. 3). In an alternative embodiment, the concave protrusion for the joint is formed in the upper plate, rather than the lower plate.

In an exemplary embodiment of the invention, each pair of corresponding slices of the two plates 101, 102, e.g., 104:115; 106:116; 108:118; 110:120; 112:122; and 114:124, has the same circumference dimension. Other designs, with different circumference dimension of corresponding slices may be provided as well.

The design of plates 101, 102 optionally enables a relative motion between them, which is intended to restore a natural mobility of the disc during spinal motion. The core element 128 and socket 141 joint design optionally permits “sliding”, rotation, twisting and/or other movements of the upper plate 102 over the core element 128 in all directions, while lateral steps 130, 132 optionally provide for natural movement limitations. Various joints as known in the art may be used. Optionally, limitations of movement in various directions are provided, for example, using a step, or using a protrusion in groove design that prevents twisting of the plates relative to each other, more than a certain amount. Optionally, one or more of the slice pairs have internal connections between the slices, for example, using cables or flexible joints, such that these cable or joints limit the freedom of motion of the joint. Optionally, the cables are used to prevent dislocating of the joint by limiting shear motion between the plates. Optionally, adjacent slices are connected together, for example, to prevent migration in the body if locking fails. Optionally, this connection is by a thread which is optionally provided after implantation, for example into a through hole connecting the slices, or by short cable sections which have a separate locking mechanism (not shown) to other slices.

The design of prosthesis 100 design can also contribute to disc height restoration, for example, by mimicking the volume of a real joint.

In embodiments where a separate “ball” element is provided, this element may be provided via a cannula, for example, as a hard rubber ball which is compressed to fit the cannula diameter and then released into matching depressions on the two plates or on only one of the plates.

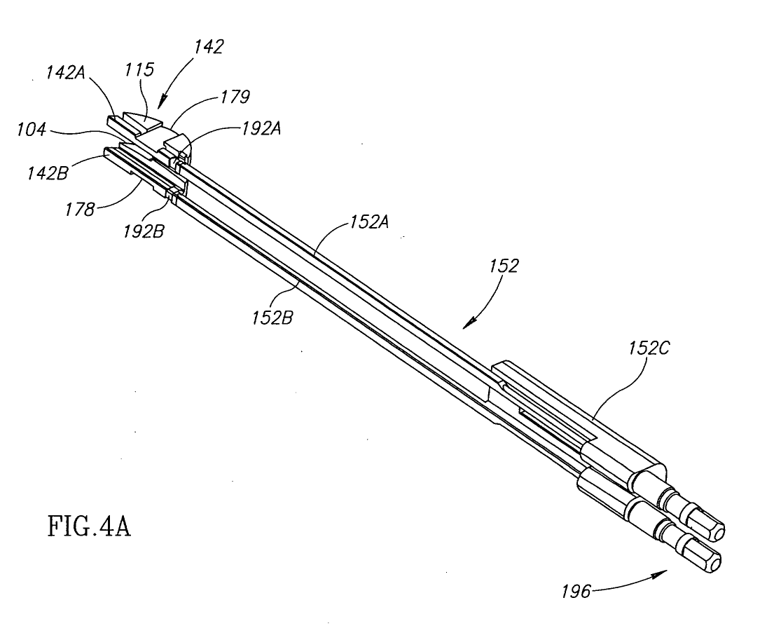

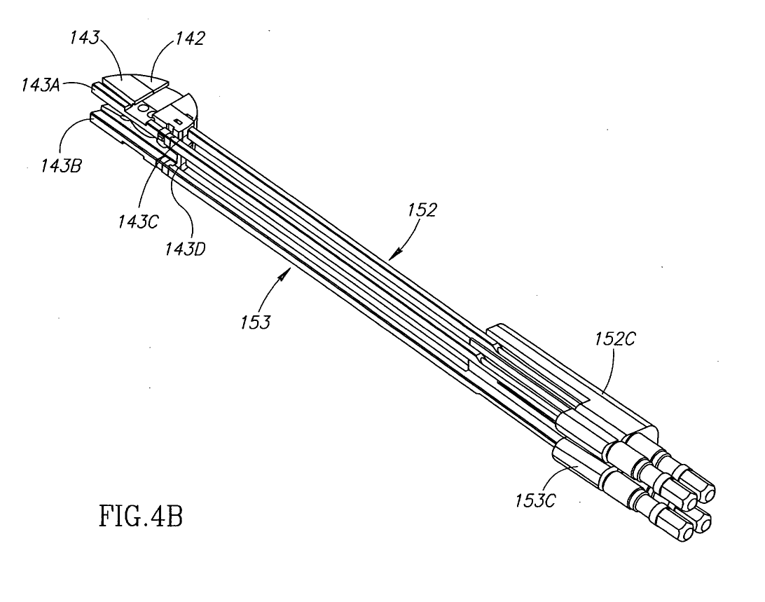

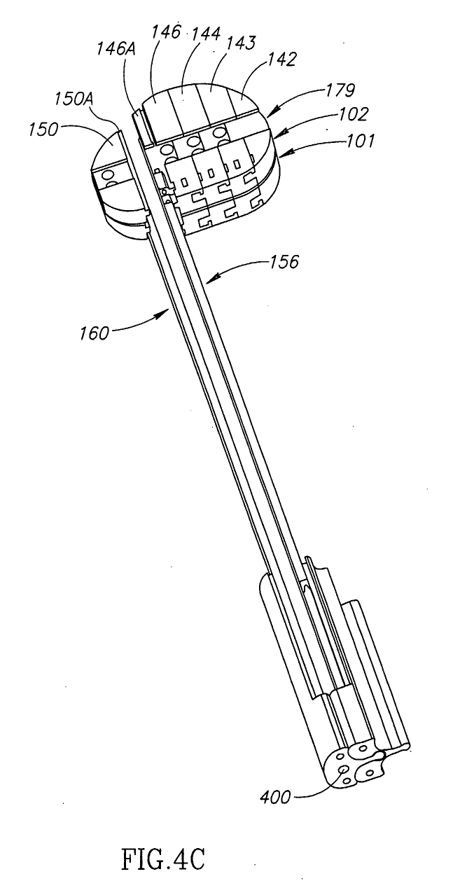

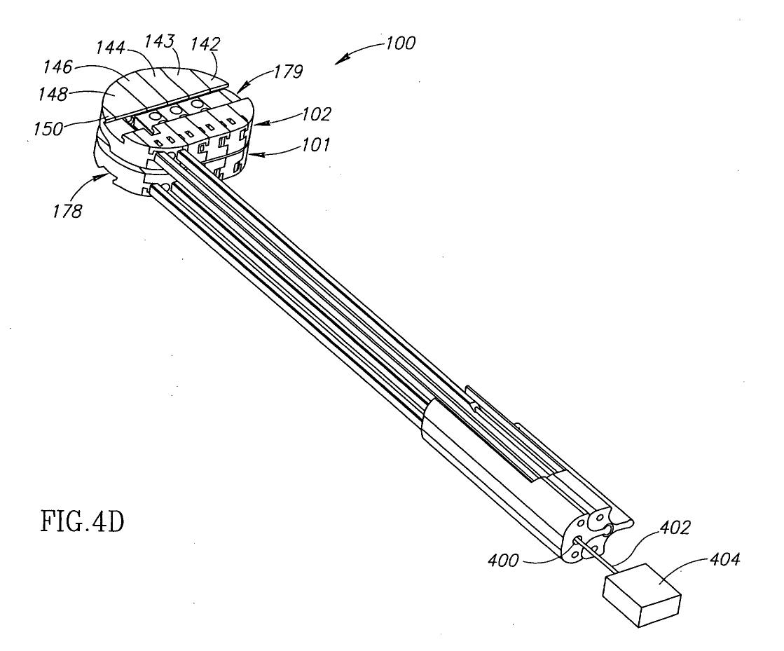

FIGS. 4A-4D illustrate stages in a process of deploying disc prosthesis 100, according to some exemplary embodiments of the invention. The completed prosthesis 100 comprises six portions 142, 143, 144, 146, 148, 150 (FIG. 4D), each including a slice of upper plate 102 and a corresponding slice of lower plate 101, which can be considered, in some embodiments, a single slice of the device. According to this exemplary embodiment, the prosthesis portions 142, 143, 144, 146, 148, 150 are provided pre-loaded on designated delivery system elements (inserters), and inserted in a consecutive order, starting with the lateral portion 142, with the optional exception that the sixth portion 150 (i.e., the portion at the other end) is introduced prior to the fifth portion 148.

In an exemplary embodiment of the invention, all six portions 142, 143, 144, 146, 148, 150 are inserted through a same port, for example, a port (e.g., an incision) of 8 mm in diameter. Other port sizes may be used, for example, selected according to the portion size. In an exemplary embodiment of the invention, the port is provided in a line with the intended location within the disc space for the fifth (or otherwise last inserted) portion of the prosthesis. Optionally, the prosthesis portions 142, 143, 144, 146, 148, 150 are connected to their inserters during the deployment rather than being pre-loaded.

The delivery systems can be, for example, 10, 20, 30 or 40 cm long. Optionally, the length is selected so that they pass through the port to the disc area but are not so long as to allow too much twisting and/or bending or other distortion of the relative position of the internal portions and the external portions. Optionally, the length is enough to reduce radiation exposure of an operating physician, by being outside a cone of radiation of a fluoroscopy system.

In an exemplary embodiment of the invention, the slices of the plates are inserted in pairs, one slice from each plate. Optionally, the pairs comprises two slices that are attached together, for example, using a permanent bond, such as an elastic section or using a biodegradable bond, such as a water solvent sugar. Alternatively, the pairs of slices are mounted together on a delivery system for being inserted simultaneously, but are not connected to each other. Alternatively, the slices are inserted consecutively.

Following removal of disc material, four portions 142, 143, 144, 146 are introduced into the intervertebral disc space as follows: the lateral portion 142 of prosthesis 100 is introduced first, loaded on a delivery system 152 thereof (FIG. 4A), and optionally slightly pushed aside toward its designated position, to enable insertion of the consecutive portion. In an exemplary embodiment of the invention, the width of the delivery system is smaller than the width of the delivered portion.

Then (FIG. 4B), the second portion 143, loaded on its designated delivery system 153, is introduced through the same port. In an exemplary embodiment of the invention, portion 142 and portion 143 interlock. In one example, portion 143 optionally has two recesses 143C, 143D at its lateral side, which match the two male elements 142A, 142B at the medial side of the first portion 142 (FIG. 4A). Thus, the second portion 143 is inserted over the medial side (male elements 142A, 142B) of the first portion 142 and the first delivery system 152. The second prosthesis portion 143 also incorporates two male elements 143A, 143B at its medial side, over which the next prosthesis 144 portion will be introduced. Optionally, the opening of the recesses are flared, to assist alignment.

In an exemplary embodiment of the invention, the delivery systems are designed so that they interconnect. Optionally, once one delivery system is inserted, the next delivery system and/or inserted portion, rides on the already inserted delivery system. In an exemplary embodiment of the invention, only the inserted portion rides. Alternatively or additionally, not all the delivery system is designed to ride. Instead, for example, only a distal portion interlocks (e.g., the “alignment portion” described below).

In one example, referring back to FIG. 4A, each delivery system, for example the first prosthesis portion delivery system 152, comprises 2 vertically parallel rods 152A, 152B, which are connected to the first portion male elements 142A, 142B. At its proximal part, the delivery system 152 comprises an external alignment section 152C for the corresponding prosthesis portion 142. This trial section 152C optionally has a design that interlocks and/or aligns with its adjacent trial section 153C. In an exemplary embodiment of the invention, the progress of assembling the prosthesis is tracked by examining the relative placement of the trial portions. Optionally, this examination is used instead of or in addition to imaging techniques.

In an exemplary embodiment of the invention, rods 152A and 152B fit in apertures formed in the “male” protrusions of the slice (e.g., 138).

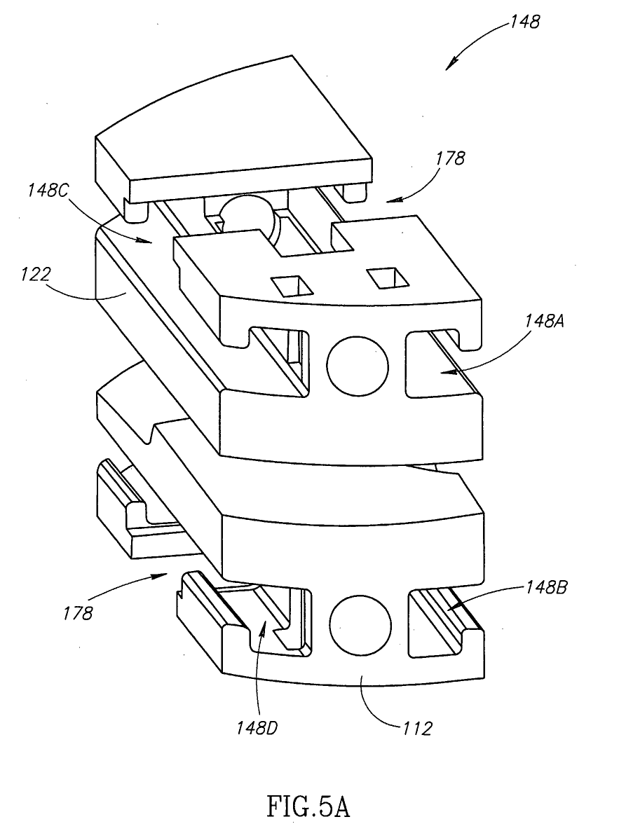

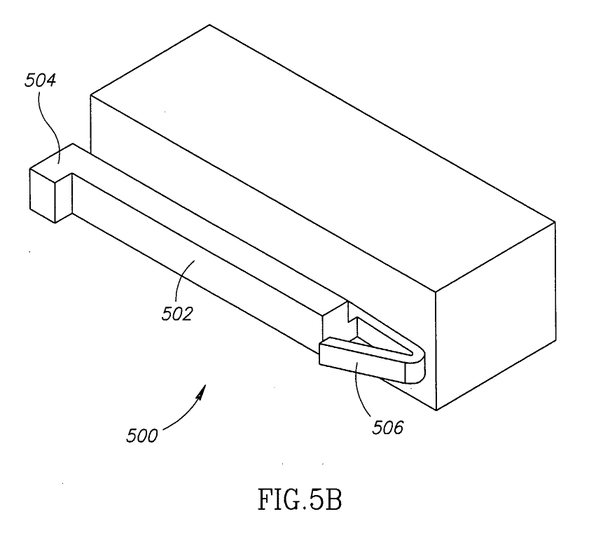

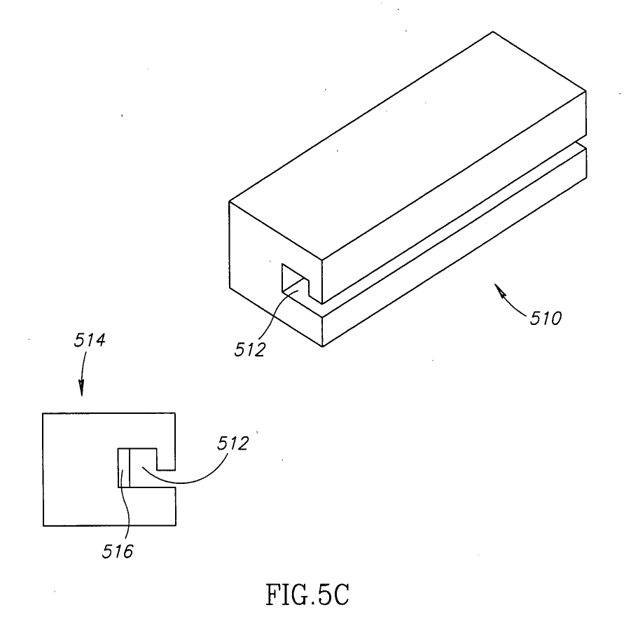

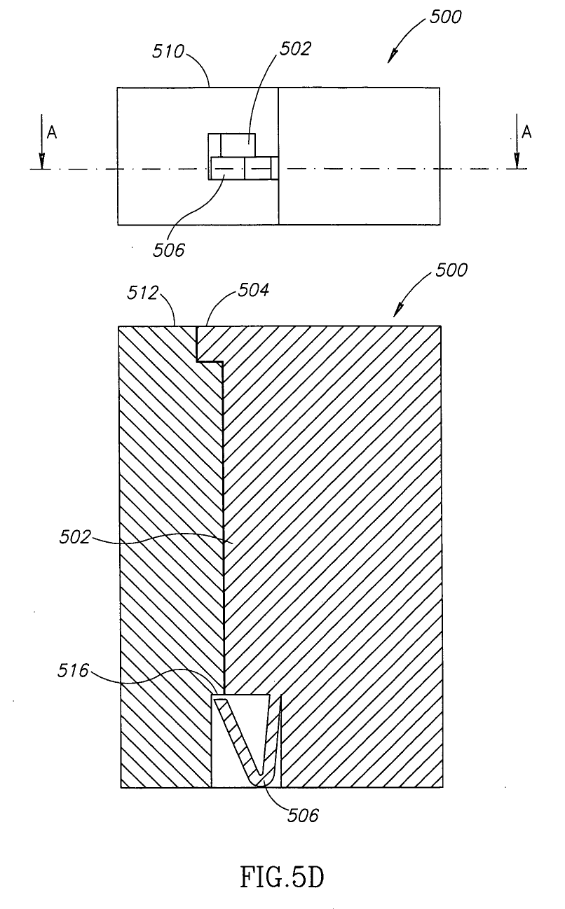

In an exemplary embodiment of the invention, the plate slices include an integral inter-locking mechanism to lock slices together, described below with reference to FIGS. 5B-5D. Alternatively or additionally, other means, for example, adhesive, are used to attach the slices together. As can be seen in FIG. 4A, each male element 142A, 142B of prosthesis portion 142 incorporates at its proximal section a pair of locking elements 192A, 192B. When the consecutive prosthesis portion, for example the second portion 143, is inserted over (“slides over”) the medial male elements 142A, 142B of the first portion 142, the locking elements 192A, 192B are pushed inside the male elements 142A, 142B, respectively, until the second portion 143 is completely inserted. Then, the locking elements 192A, 192B are facing matching grooves (not shown in FIG. 4) at the lateral side of the second portion 143 and enter said grooves. This locking mechanism optionally prevents anterior-posterior relative motion between adjacent prosthesis portions.

FIGS. 5B-5D illustrate, in a simplified manner, a locking mechanism suitable for the slices described above, in accordance with an exemplary embodiment of the invention.

FIG. 5B shows a male block 500 (a simplification of a male link section of a slice), includes an elongate rail 502, an optional end stop 504 (which may be replaced by a locking element) and a locking element 506, for example, a folded leaf having a resting condition in which it extends past rail 502. Optionally, locking element 506 is elastic. Alternatively or additionally, a super-elastic or shape memory material may be used. Optionally, cooling or heating is used to release locking element 506, if needed. Other types of interlocking mechanism can be used.

FIG. 5C shows a female block 510 including a channel 512. In a cross-sectional view 514, a locking area 516 is shown.

FIG. 5D shows an interlocked configuration, showing the locking of male block 500 to female block 510 via stop 504 and interlocking of locking element 506 and locking area 516 (e.g., a step)

As shown, rail 502 has a “P” cross-section. However, other cross-sectional shapes can be used. In particular, some rotation and/or axial and/or trans-axial freedom may be allowed. In one example, rail 502 has a rounded cross-section. Similarly locking element 506 may be thinner.

Following insertion of the second portion 143, the delivery system 152 of the first portion 142 is optionally detached and removed. Optionally, said detachment is by unscrewing a screw 196 at delivery system proximal part. Optionally, the screw is long enough to reach to the portion and engage a threaded aperture therein. Other detaching methods may be used as well. In one example, an inner rod inside each delivery system rod 152A, 152B connects the delivery system to the prosthesis, and by unscrewing a nut at delivery system proximal end, the inner rod may be rotated until it is sheared (not shown in FIG. 4). In another example, the rod holds the locking element from either side thereof. When pulled back, this releases the holding. Optionally, the rod comprises a pinch-gripping element, which is released, for example, by retracting a sheath that urges two gripping jaws towards each other. In another example (not shown) the locking element includes a hole into which the rod is threaded.

After the two portions are implanted and assembled, the assembled portions 142, 143 are pushed slightly aside, and the third prosthesis portion 144 and afterwards the fourth prosthesis portion 146 are similarly inserted into the inter-vertebral space, loaded on their delivery systems 154, 156, respectively, and assembled.

With regard to completing the assembly of the prosthesis, in an exemplary embodiment of the invention, the last portion inserted is the ordinally last one. Alternatively, and as shown in FIG. 4, the next to last portion is skipped and the (spatially) last portion is inserted before it. Optionally, this allows the final assembly and alignment to be between artificial elements and not between an artificial element and surrounding flesh which might press directly on the last element. Optionally, as noted above, the port is aligned with the window in the prosthesis into which the (temporally) last portion is inserted. Optionally, the prosthesis is assembled from more than two sub-assemblies and/or the two sub-assemblies shown in FIG. 4 each consist of more than one slice/portion. Optionally, different ports are used for the different sub-assemblies. Optionally, the ports comprise flexible tubes pushed through the flesh, for example, using cannulae, rather than incisions.

In an exemplary embodiment of the invention (FIG. 4C, in which the two upper rods of the two delivery systems are not shown), a sixth portion 150 is introduced, loaded on its delivery system 160, through the same port, and skipping the fifth (or otherwise numbered next to last portion). Sixth portion 150 is then slightly pushed to the other side, to its intended position. Optionally, unlike the previous inserted portions 142, 143, 144, 146, all of which having male elements 142A-B, 143A-B, 144A-B, 146A-B and delivery system 152, 153, 154, 156 at their same side (directed medially), the male elements 150A-B and delivery system 160 of the sixth portion 150 is located at the opposite side (also medially), to allow for locking of the fifth and sixth portions 148, 150, respectively, together. It should be noted that the delivery systems are at extreme ends of the “missing” slice. In an exemplary embodiment of the invention, the missing slice has female-female connections on it, as the male connection on the fourth and sixth slices is attached to the delivery system and it is desirable in some embodiments that the delivery system be attached as near to the edge of the slice as possible. Not shown for clarity, and described below in FIG. 4D, is a delivery system and portion 148, which are optionally provided pre-mounted on delivery system 160, but only advanced after portion 150 is in place, in some embodiments of the invention.

Alternatively, a male and female or a double male section is used for the final portion. In an exemplary embodiment of the invention, the selection of which slice to insert last depends on anatomy and availability of direct access to the implant area (e.g., intervening nerves and dura).

In an exemplary embodiment of the invention, in a same plate, only one male-female link is used to link two nearby slices. Optionally, two or more such links are provided. Alternatively or additionally, the links are flexible enough to allow bending of the link between the slices, for example, to less than 30 degrees, less than 10 degrees, less than 5 degrees or smaller or intermediate amounts.

Optionally, the slices are inserted into a retraining bag. In an exemplary embodiment of the invention, the implantation process starts by implanting a bag and then the slices are inserted into and assembled inside the bag. Optionally, the bag is used to prevent migration. Optionally, a bag is provided per plate and/or per disc. Optionally, the bag includes an aperture for the joint area.

The last portion introduced is a fifth portion 148 (FIG. 4D). Fifth portion 148, shown in FIG. 5A, has at each side of its plate slices 112, 122 female recesses 148A, 148B, 148C, 148D that match the male elements 146A, 146B, 150A, 150B of its adjacent portions 146, 150. As the fifth portion/slice may not include male portions to attach the delivery system to, the parallel rods of the fifth portion delivery system may be connected to the fifth portion 148 at a different place comparing other portions 142, 143, 144, 146, 150 for example at the center of the portion slices 112, 122. In an exemplary embodiment of the invention, portion 148 is provided outside the body before or with portion 150, however, it is not advanced until portion 150 is in place. Optionally, the path of portion 148 is constrained by the rods of the delivery systems of portions 146 and 150, which rods are inside the female grooves of portion 148 (see FIG. 5A). In FIG. 4D, the delivery rods for portion 148 are not visible, being between the other delivery rods and removed for clarity.

In the embodiment shown, an aperture 400 is formed in the handle of delivery system 160 of portion 150. After portion 150 is in place, a handle 404 can be advanced and pushing a rod 402 via aperture 400, advance portion 148 to lock as shown. Optionally, handle 404 interlocks with the handles for systems 160 and 156. In the figure, for clarity, handle 404 is shown not completely advanced, even though portion 150 is already in place. Optionally, the interlocking of handle 404 with systems 160 and 156 is different from the other interlockings of the external sections. Optionally, aperture 400 is large enough so that the interlocking resembles that of portion 148 with portions 146 and 150.

Optionally, the prosthesis portions are introduced in a different sequence than the one described above, and their design, with respect to male-female locking mechanism design and delivery system location may be modified respectively.

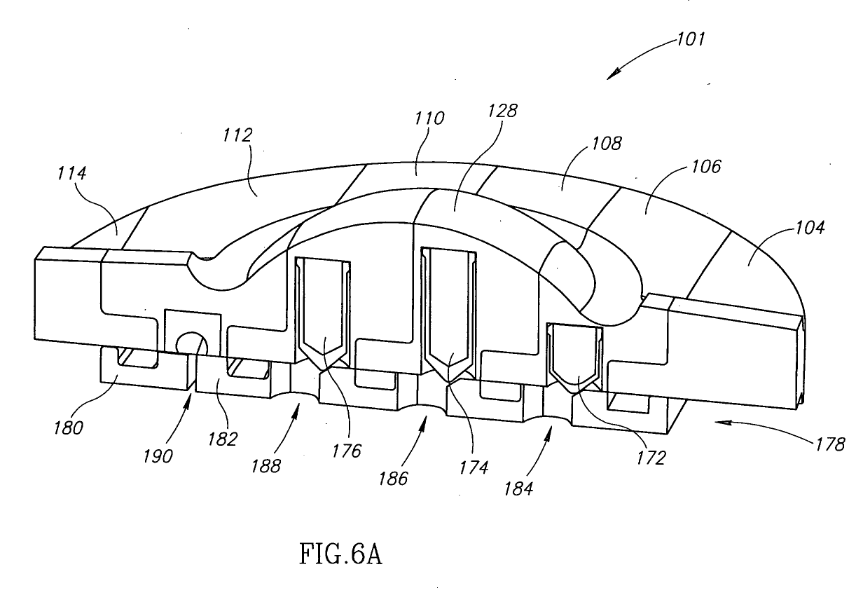

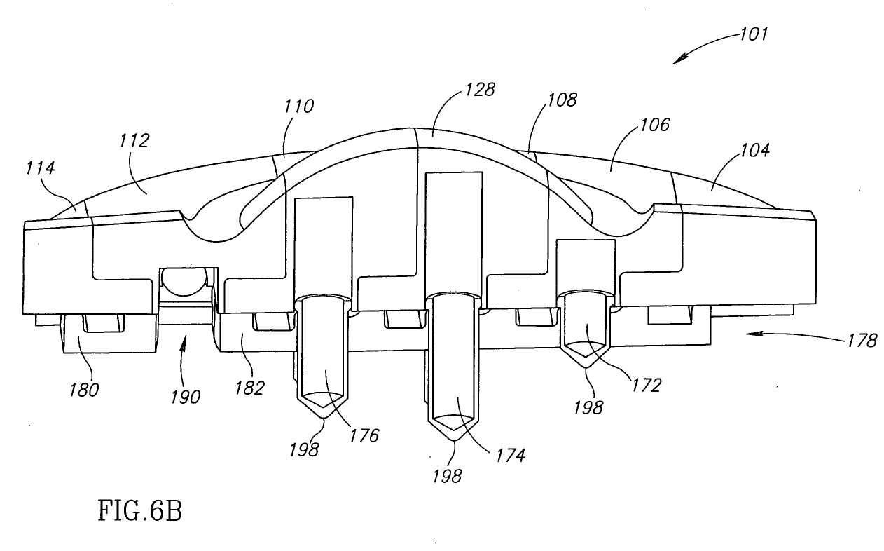

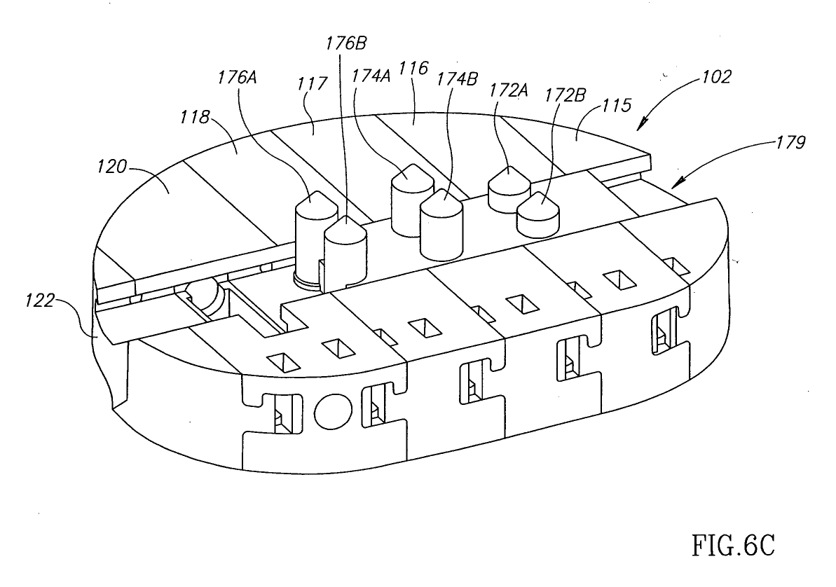

FIGS. 6A-6C illustrate a method of connecting of prosthesis 100 to its adjacent vertebral endplates, for example, in order to prevent prosthesis migration, in accordance with an exemplary embodiment of the invention. In the method shown in these figures, a plurality of protrusions pop-out of prosthesis 100 once it is fully assembled. Optionally, these protrusions pop-out automatically when assembly is complete. Alternatively, the popping-out maybe actuated manually. Alternatively, protrusions may be popped-out as the assembly progresses.

FIG. 6A is a cross section of prosthesis inferior plate 101. Three vertical spring-loaded spikes 172, 174, 176 are located inside the interior of the plate 101 (springs not shown). Other number of spikes with different dimensions, contact surfaces, numbers and/or location may be used as well. Plate 101 also optionally comprises a horizontal windowed element 178 (or set of elements, one for each portion) beneath the spikes 172, 174, 176. In an alternative embodiment, a fixed slot 178 is provided on which the track elements are mounted and may move. In an alternative embodiment, the track elements are part of the delivery system and removed with it.

Windowed element 178 optionally incorporates two (or fewer or more) track elements 180, 182. An optionally large track element 182 includes three holes 184, 186, 188, each of which is positioned, during insertion phase of prosthesis portions 142, 143, 144, 146, 148, 150, beneath and adjacent to the three spikes 172, 174, 176. When desired, optionally following insertion of prosthesis portions 142, 143, 144, 146, 148, 150 assembly, track elements 180, 182 are pushed to opposite sides (see below) to reposition the three holes 184, 186, 188 exactly beneath the three spikes 172, 174, 176. This enables spikes 172, 174, 176 to penetrate through holes 184, 186, 188 into or against the adjacent vertebral endplate (FIG. 6B).

Alternatively or additionally to protrusions, flow of an adhesive or a bioactive chemical is enabled when windowed element 178 moves, thereby adhering the plates to the vertebral plates.

In an exemplary embodiment of the invention, a gap 190, for example a conic-shaped gap, separates the two tracks 180, 182 at the last inserted portion 148 of prosthesis 100. Upon insertion into the gap 190 of a screw, optionally incorporated at the distal end of last portion delivery system 158 (and optionally advanced only when interlocking between the slices is completed), track elements 180, 182 are slightly pushed aside, and the spikes 172, 174, 176 protrude. A penetrating end 198 of the spikes 172, 174, 176 is optionally conical or edged, to facilitate spikes penetration into endplates. FIG. 6C illustrates similar spikes 172A-B, 174A-B, 176A-B of the upper plate 102 (in a penetration state).

In an automatic embodiment, a protrusion (not shown) on the last inserted slice engages and moves the track elements (e.g., via gap 190). Optionally, the track elements of adjacent slices interlock when pushed sideways by the last slice. Then, forward movement of the protrusion on the last slice aligns the apertures and the spikes.

In an alternative embodiment, the delivery system for each portion physically prevents movement (e.g., by blocking) of the spikes, for example using a third rod (or the existing rods) with a spatula-like extension that fits under the slice, and when removed, the spikes protrude and/or penetrate. Alternatively or additionally, rotation of the rod of the delivery system rotates the spike, for example there being a matching protrusion and/or threading on the spike and the rod, which rod when rotated advances.

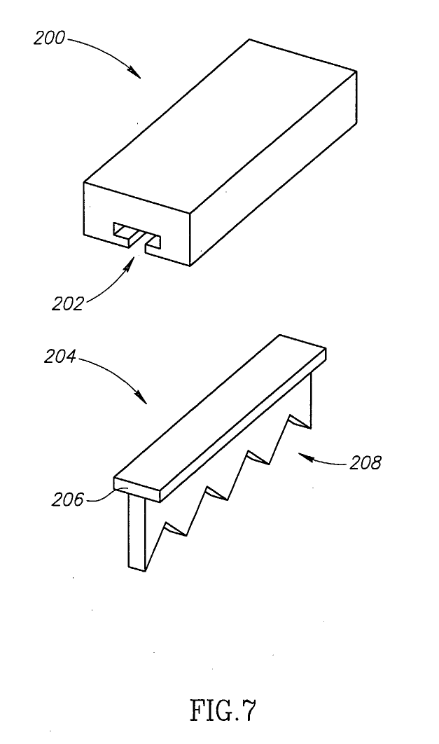

FIG. 7 shows another option for connection between prosthesis plates 101, 102, and adjacent vertebral endplates, in accordance with an exemplary embodiment of the invention. A slice 200 of prosthesis 100 is generally illustrated, in a simplified manner. Slice 200 comprises at its surface that contacts the vertebral endplate a slot 202, into which a horizontal section 206 of a protrusion section 204 is inserted, optionally hammered, following introduction of said plate slice 200 (together with its corresponding slice of the other plate). After assembly, one or more protrusion teeth 208 penetrate or abut the vertebral endplate. Optionally, slice 200 is held using its delivery system during insertion of protrusion section 204. Additional pins may be inserted as well to prevent anterior-posterior movement of prosthesis (not shown in FIG. 7). Alternatively or additionally, section 204 snap-locks to slot 202. Optionally, teeth 208 are flexible.

In an exemplary embodiment of the invention, a plate (101, 102) has a thickness of between 1 and 20 mm, optionally about 7 mm. Other sizes, for example, to match physiological needs, maybe used. Optionally, a plate is formed of layers, each layer being assembled from slices.

In an exemplary embodiment of the invention, the diameter of the disc implant is about the same size as an excised disc. Optionally, an oversize, of for example, 10% or an undersize, of for example, 10%, 20% or more may be provided. Size considerations as common in the art of disc replacements may be used. It is noted however, the limitations of sizes due to implantation routes need not apply. Optionally (not shown), the edges of the plates are selected to cap the vertebral end plates, optionally reaching around the vertebra (e.g., along the spine), for example, at one or more points or as a rim. Optionally, a depression to protect the spinal column is provided.

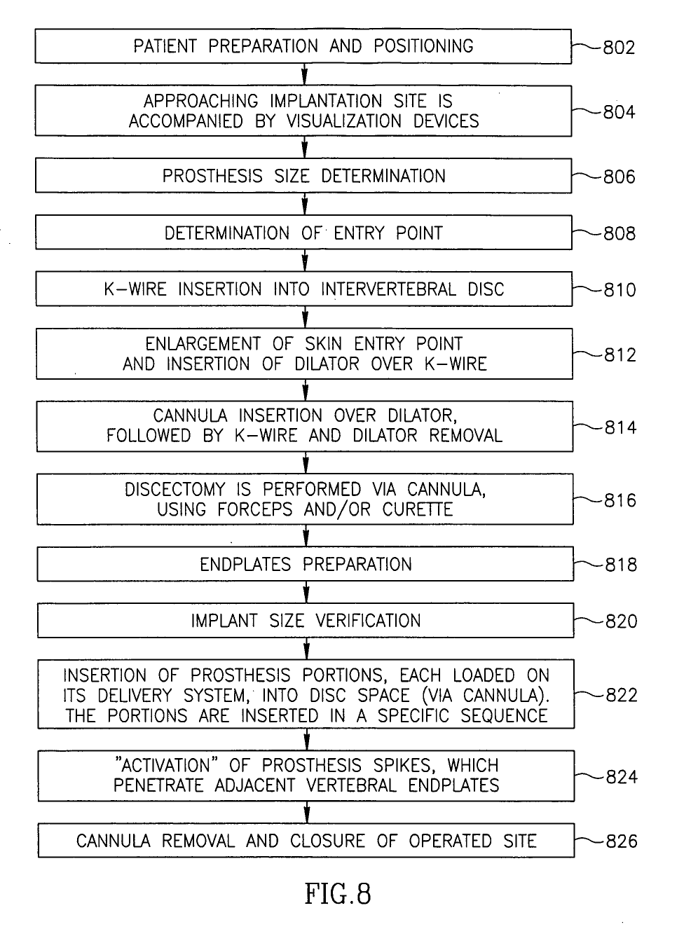

A general, typical description of the method described above is presented in the FIG. 8, in accordance with some exemplary embodiments of the invention. The operation may be performed in a percutaneous posterior approach.

At 802, the patient is prepared.

At 804 the implantation site is optionally imaged.

At 806, a prosthesis size is optionally estimated.

At 808, an entry point is selected, for example, to prevent/minimize damage to tissue and/or to match an order of implant slice insertion. An implant may be selected to match possible approach directions.

At 810, a K-wire is optionally inserted to the implant area.

At 812, the skin entry point is optionally enlarged by a dilator over the K-wire.

At 814, a cannula is optionally inserted over the K-wire, forming a port.

At 816, discectomy is optionally performed via the cannula, for example using methods known in the art.

At 818, the endplates are optionally prepared, for example, by eroding, for example, to induce bleeding. Optionally, damaging of the plate is reduced or avoided, to prevent ossification of the joint.

At 820, the size of required disc, for example, diameter and/or height are optionally determined.

At 822, the portions are inserted in series, for example as described above.

At 824, the spikes are optionally activated to engage the end plates.

At 826, the cannula is removed.

Optionally, no cannula is used. Instead, each portion may be streamlined to avoid tearing tissue on its inward journey. Optionally, the prosthesis portions and/or delivery systems ride on a K-wire.

Optionally, the cannula used is a square- or rectangular-lumen cannula or otherwise matches the cross-section of the inserted slices. A matching dilator may be used as well.

In a removal process, the attachment of the slices to the endplate is optionally broken, for example, by vibration. Optionally, the protrusions are released by tearing the springs, for example, via an axial access hole (not shown) to the spring chamber). Optionally, a thread is attached to the back side of the protrusions and passes through such an access hole or is capable of being grabbed there-through. Then, pulling on the thread pulls back the protrusion.

Each slice is optionally removed by attaching a delivery system thereto and releasing the sliding locks used to interlock slices. Optionally, the locks are released by drilling through the locking elements, through an axial access hole provided therefore. Optionally, the locking element is designed to release when an element is pushed against it (e.g., via the access hole). In an alternative embodiment, the slices are removed in an order in which the relevant locking elements are on the side of the disc near the delivery system, in which case the delivery system can be used to directly compress the locking element to release the locking. In embodiments where the delivery system is attached to male clink portions, an extension to the female link element, where the locking element is engaging, is provided.

The term axial is used to describe a hole along the long dimension of the slices, generally the insertion direction.

Optionally, the axial access hole(s) are used for attaching of the delivery system thereto.

Optionally, once the delivery system is attached to a slice, the slice is moved sideways (with the rest of the disc) to be opposite the port and then pulled out. Optionally, the next delivery/removal element is attached before the slice is removed.

It is noted that all the above-mentioned components are not restricted to the above-mentioned dimensions. Said dimensions are typical, and may vary and/or become part of a range of dimensions.

In an exemplary embodiment of the invention, the delivery systems and slices are provided as a kit. Optionally, several sizes of implants are provided. Optionally, a kit is sterile and may include instructions for use, for example, for the above methods.

In an exemplary embodiment of the invention, the apparatus and methods described herein are used for partial disc replacement, for example, for providing a new vertebral plate.

Various features of device and method have been described. It should be appreciated that combinations of the above features are also considered to be within the scope of some exemplary embodiments of the invention, as are embodiments which include fewer than all the features described above. It should also be appreciated that some of the embodiments are described only as methods or only as apparatus, however the scope of the invention includes both methods for using apparatus and apparatus for applying methods. The scope of the invention also covers machines for creating the apparatus described herein. In addition, the scope of the invention also includes methods of using, constructing, calibrating and/or maintaining the apparatus described herein. When used in the following claims or in the text above, the terms “comprises”, “comprising”, “includes”, “including” or the like mean “including but not limited”.

1. An artificial disc device, comprising a plurality of interconnected elements arranged in a row, adapted to be assembled in situ so that at least two of the elements of the row contact a same vertebral endplate.

2. (canceled)

3. A device according to claim 1, wherein said elements are adapted to interlock at least in pairs.

4. An artificial disc devices comprising at least a first row of interconnected elements and a second row of interconnected elements, each of the first row and the second row comprising a plurality of the elements adapted to be assembled in situ;
wherein the first row and the second row are each adapted to be placed near an opposite vertebral plate, along the axis of the spinal column of a patient into whom it is inserted so that each of the first row and the second row form one of a pair of opposing disc plates.

5. A device according to claim 4, wherein said plates define a ball joint between them.

6. A device according to claim 5, wherein said ball joint comprises a ball and socket and wherein said ball is integral to one of said plates.

7-8. (canceled)

9. A device according to claims 3, comprising at least one protrusion adapted to selectively deploy and engage a vertebral plate.

10. A device according to claim 9, wherein said protrusion is adapted to deploy when said deployment is complete.

11. A device according to claim 9, wherein said protrusion self-deploys.

12. (canceled)

13. A device according to claim 3, wherein said interlocking elements, after said interlocking have some freedom of relative motion.

14. A device according to claim 13, wherein said elements interlock using a self-locking mechanism.

15. A device according to claim 3, wherein said interlocking elements comprise alignment portions in the form of matching parts.

16. A device according to claim 15, wherein said alignment portions comprise male-female matching portions.

17. A device according to claim 1, comprising a delivery system attached to at least two of said elements.

18-20. (canceled)

21. A device according to claim 4, wherein said members are formed of metal.

22. A device according to claim 1, having an osteointegrating outer layer.

23. A method of deploying a spinal disc prosthesis, comprising:
forming a path to a disc area;
providing a first disc portion to said disc area;
providing a second disc portion to said disc area; and
assembling said second disc portion to said first disc portion so that the portions are arranged in a row at said disc area so that at least two of the portions contact a same vertebral endplate.

24. (canceled)

25. A method according to claim 23, wherein said providing a first disc portion and said providing a second disc portion comprises aligning delivery elements each associated with one of said disc portions.

26. A method according to claim 25, comprising removing a delivery element associated with said first portion, but not an element associated with said second portion, when a third disc portion is provided using a third delivery element.

27. A method according to claim 23, comprising releasing at least one protrusion to contact an adjacent vertebral plate.

28. A method according to claim 27, wherein said release is after performed said assembly.

29. A method according to claim 27, wherein said release is performed during said assembly.

30. (canceled)

31. A method according to claim 23, wherein said path is to a side of a dura.

32. A method according to claim 23, wherein said path is for posterior access.

33. A method according to claim 23, wherein said path has a maximum diameter of less than 20 mm.

34. A method according to claim 23, wherein said path has a maximum diameter of less than 15 mm.

35. A method according to claim 23, wherein forming said path comprises inserting a cannula for said providing.

36. A method according to claim 23, wherein assembling comprises interlocking.

37-45. (canceled)

46. A method according to claim 23, repeated so that at least two rows are formed.

47. A method according to claim 23, wherein assembling includes sliding one of the portions with respect to another of the portions.

48. A method according to claim 23, performed so as to maintain a capacity for relative motion between plates.

49. A method of assembling a spinal disc prosthesis within body, the method comprising:
(a) creating a space between opposing first and second vertebral endplates;
(b) assembling a first disc level opposing the first vertebral endplate within said space;
(c) additionally assembling a second disc level opposing the second vertebral endplate within said space;
wherein each of said assemblies comprises assembling at least two interconnectable elements in a row.

50. A method according to claim 49, wherein the assembly comprises a sliding engagement.
